(19) United States
(12) Patent Application Publication
(10) Pub. No.: US 2007/0129594 A1
(43) Pub. Date: Jun. 7, 2007

(54) Implantable direct cardiac compression device and system

(75) Inventors: Yifei Huang, New South Wales (AU); Stephen Nicholas Hunyor, New South Wales (AU)

Correspondence Address: WOOD, HERRON & EVANS, LLP, 2700 CAREW TOWER, 441 VINE STREET, CINCINNATI, OH, 45202, US

(73) Assignee: HEART ASSIST TECHNOLOGIES PTY LTD, New South Wales (AU)

(21) Appl. No.: 10/567,520

(22) PCT Filed: Aug. 12, 2004
(86) PCT No.: PCT/AU04/01073

§ 371 Date: Feb. 9, 2007

(30) Foreign Application Priority Data
Aug. 12, 2003 (AU) 2003904283

(51) Int. Cl. A61M 1/10

(52) U.S. Cl. 600/16

(57) ABSTRACT

An implantable direct cardiac compression (DCC) device (101) has a body (102) comprising a flexible frontal cardiac compression wall (103) and a rear wall (104) together defining a pressurisable chamber (106). The cardiac compression wall (103) is adapted to be affixed to the wall of a ventricle (21) of a heart (20) and to compress the ventricle (21) upon pressurisation of the chamber (106). The device (101) is provided with two flexible flaps, one extending from each of two of opposing lateral sides of the body (102), adapted to be affixed to the ventricle wall (23). An implantable DCC system comprises left and right DCC devices (101, 201) for affixing to the left and right ventricles (21, 22). Straps (216) are provided on the right DCC device (206) for wrapping around the heart (2) and left DCC device (101) to secure the DCC devices (101, 201) to the left and right ventricles (21, 22) respectively.

### FIELD OF THE INVENTION

The present invention relates to heart assist devices, and particularly relates to an implantable direct cardiac compression device and system.

### BACKGROUND OF THE INVENTION

Heart failure is a clinical condition with symptoms that include shortness of breath, lack of energy and swelling of ankles. Progressively more severe heart failure further degrades health through severe weight loss, muscle wasting, failure of other organs (particularly the kidneys or liver), compromised immune response and risk of infection. Heart failure is widespread in the community, requiring frequent medical attention with associated high medical costs, estimated to be US$20-40 billion in the USA in 2000. The incidence of heart failure doubles every decade after the age of 45 and its prevalence has risen steeply as a result of improvements in clinical management of heart attack and coronary artery disease, leaving surviving patients with long term heart damage.

Heart failure is triggered by impairment of the pumping function of heart muscular tissue, leading to reduction in blood supply to tissues and organs and thereby the supply of nutrients, particularly oxygen, to the body. The body responds to this situation by activating compensatory nervous system and hormonal mechanisms that have detrimental side effects. Further, the body's cells produce damaging inflammatory proteins such as cytokines. The disorder of heart muscle function associated with heart failure results predominantly from coronary artery disease and/or heart attack.

Current treatments for heart failure are based predominantly on drugs. However, some drugs have been found to be ineffective or even harmful, and drug therapy is of limited benefit for heart muscle that is severely damaged. Other current treatments include cardiac resynchronisation therapy and heart transplantation. There is also intensive research in the fields of cellular and gene therapies although their clinical application seems remote. Each of these treatments, however, suffer from various limitations and difficulties.

Alternative therapies involve some form of mechanical heart support. Most current mechanical heart assist devices tap into the blood stream and act to directly circulate the blood, thus complementing or replacing the heart's pumping function. Such blood contacting devices can be characterised as either non-pulsatile flow devices, which use centrifugal/rotary or axial flow (turbine) pumps to produce a non-pulsatile flow, or pulsatile flow devices, which use hydraulic, electromechanical or pneumatic means to provide the more physiological pulsatile type of flow. These blood contacting devices, however, suffer from various deficiencies, particularly the occurrence of blood clotting when the blood contacts the device and the possibility of hemorrhage and septicemic blood infections.

As a result of these deficiencies of blood contacting heart assist devices, non-blood contacting heart assist methods provide some potential advantages. Such non-blood contacting heart assist methods include passive surround devices such as that disclosed in U.S. Pat. No. 5,703,343 which act as containment devices for failing hearts, but which are incapable of actively augmenting the heart's pump function.

A preferred non-blood contacting heart assist method is cardiac compression, which in its most simple life-saving form has been used for many years and involves the compression of the chest wall of a patient. Direct cardiac compression (DCC) methods, whereby compression is applied directly to the heart, originated with surgeons manually compressing a severely compromised or arrested heart during emergencies that may occur in open-chest operations. More recent DCC methods include cardiomyoplasty, which involves mobilising a powerful chest wall muscle (the latissimus dorsi), introducing it into the chest cavity and using it to cradle the heart. The muscle is then electrically stimulated in synchrony with the heart so as to provide pumping support. However, this method has not experienced any significant success in clinical use.

Most current DCC methods involve the implantation of active devices which at least partially surround the left and right ventricles and are activated to compress the ventricles during systole (ventricular contraction), thereby assisting contraction of the ventricles to eject blood. The devices are deactivated for diastole (ventricular relaxation), aiming to allow the ventricles to relax for filling (“priming”). Early examples of such devices, which have not displayed significant sustained improvement of heart function, are disclosed in U.S. Pat. Nos. 3,455,298 and 5,713,954.

Another form of implantable direct cardiac compression system, with potentially superior performance when compared to previous direct cardiac compression devices, is described in WIPO International Publication No. WO 00/78375, the disclosure of which is incorporated herein by cross-reference. This publication describes an implantable direct cardiac compression system called the “HeartPatch Pump”. An example of one DCC patch device 1 of a HeartPatch Pump is depicted in FIG. 1 of the accompanying drawings with FIG. 2 depicting a HeartPatch Pump comprising two such DCC patch devices 1a, 1b attached to a failing heart 20.

Each DCC patch device is in the form of a patch-like body 2 having a flexible frontal cardiac compression wall 3 and a less flexible rear wall 4 bounding a pressurisable chamber which communicates with the internal duct of a tube 5 secured to the longitudinal end of the body 2. The rear wall 4 of the patch-like body may achieve its lower flexibility characteristics by the provision of a reinforcing mesh which stiffens the rear wall 4 as compared to the flexible cardiac compression wall 3 such that pressurisation of the body chamber, inflating the body 2, displaces the more flexible cardiac compression wall 3 away from the rear wall 4.

For implantation of the HeartPatch Pump, an incision is made in the chest wall to access the chest cavity. Subsequently, the pericardium which encases the heart, is opened and the left and right DCC patch devices 1a, 1b are introduced through the incision, ideally in a closed configuration within a cannula or some other form of delivery device using a minimally invasive endoscopic procedure. The right patch device 1b is then located in position within the pericardial space engaged with the wall of the right ventricle 22. The left patch device 1a is positioned engaged with the wall of the left ventricle 21, which is located generally on the anterior side of the heart 20. The outer surface layer of the cardiac compression wall 3 of each of the patch device bodies 2 is formed of a porous biointegratable material, which may typically comprise woven TecoFlex ™ mesh, Seare Biomatrix™ or Gore-Tex Dual Mesh Biomaterial™ but not be limited to these materials. The biointegratable material may also use any form of tissue scaffold, including collagen, and may be seeded with various cellular elements. The biointegratable material integrates with the epicardium or outer layer of the heart wall by the ingrowth of vascularised cellular tissue. Such biointegration, however, typically takes approximately one week to provide a sufficient degree of attachment of the patch devices to the heart.

The pressurisation tubes 5 of each patch device extend through the incision made in the pericardium and chest to a pressurisation and control mechanism that may be located inside the body or external to the body. The body 2 of each patch device incorporates an ECG electrode to detect the electrical activity of the heart and which is coupled to the control mechanism. The control mechanism acts to pressurise body chambers during systole so as to assist contraction of the ventricles 21, 22 and to de-pressurise the chambers during diastole so as to enable unrestricted relaxation of the ventricles 21, 22. The HeartPatch Pump remains idle when the control mechanism determines that no cardiac assistance is required, thereby saving power.

The patch body cardiac compression walls 3 may also be fitted with sonomicrometer piezoelectric sensors which measure dimensions of the heart and deflection of the cardiac compression walls 3. Feedback from these sensors can be used by the control mechanism to tailor operation of the HeartPatch Pump as required. Use of the sensors is described in WIPO International Publication No. WO 02/065908, the disclosure of which is incorporated herein by cross-reference.

One problem associated with the current HeartPatch Pump is the lack of acute attachment to the heart whilst biointegration takes place. When closing the incision created by the operation, the opening in the pericardium, which typically consists of a vertical slit, is not closed off as the added bulk of the patch devices 1a, 1b within the pericardial space prevents closure of the incised pericardium without creating a constriction of the heart 20 which would inhibit relaxation of the left and right ventricles 21, 22. The pericardium is also left open to enable the egress of any fluids which may, accumulate as a result of any irritation inside the pericardium space resulting from the implantation of the HeartPatch Pump, although this has not been a significant issue to date. Accordingly, whilst the intact rear of the pericardium provides some support to the right patch device 1b located on the posterior side of the heart 20 whilst the biointegration process takes place, little support is provided by the open region of the pericardium located on the anterior side of the heart over the left patch device 1a.

Whilst the disclosure of International Publication No. WO 00/78375 proposes the use of an elastic mesh placed around the heart 20 and over the patch devices 1a, 1b to initially secure the patch devices in place whilst biointegration takes place, the mesh constricts across the extent of the ventricles inhibiting relaxation during diastole.

As a result of the delay in biointegration of the patch devices, and the difficulty in adequately securing the device against the heart wall in the interim, the HeartPatch Pump is typically not activated until sufficient biointegration has occurred, typically approximately 1 week after implantation. Whilst this may be satisfactory for the long term treatment of patients experiencing gradual deterioration of the heart function, it does not provide a short term solution for emergency patients presenting in an acute condition, such as following a massive heart attack, requiring immediate cardiac assistance. There is thus a need to provide for acute attachment of the implantable direct cardiac compression device, enabling immediate activation of the device upon implantation.

A further potential problem associated with the HeartPatch Pump, and particularly the left patch device 1a, is delamination of the edges of the cardiac compression wall 3 from the left ventricular wall 23. This problem is discussed with reference to FIGS. 3 and 4 of the accompanying drawings, depicting a cross-section of a left patch device 1a attached to the left ventricle 23 and being in non-pressurised (during diastole) and pressurised (during systole) states respectively. Pressurisation of the body chamber 6 deforms the cardiac compression wall 3 away from the rear wall 4 against the left ventricular wall 23. Whilst expansion of the body chamber 6 produces a compressive load across most of the interface between the cardiac compression wall 3 and the left ventricular wall 23, tensile peel stresses are created at this interface toward the lateral edges 3b of the cardiac compression wall 3. This is a result of the ballooning effect of the cardiac compression wall 3 whereby the central portion 3a gradually moves from a concave unpressurised state (shown in FIG. 3) toward a convex pressurised state (shown in FIG. 4) pushing into the left ventricular wall 23, whilst the lateral edge portions 3b tend to pull away from the left ventricular wall 23. The tensile peel stresses so created tend to delaminate the cardiac compression wall lateral edges 3b. The convexity or bowing of the cardiac compression wall 3 of the inflated patch device further increases once delaminated, which tends to cause strutting of the underlying native myocardium of the left ventricular wall 23, resulting in unusual deformation of the left ventricle 21.

Delamination and bowing are of particular concern for the left patch device since the left ventricle operates at a higher pressure as such is required to pump oxygenated blood throughout the body whereas the right ventricle only pumps de-oxygenated blood to the lungs. The left ventricular wall 23 also has a smaller radius of curvature than the right ventricle 22, further exacerbating the delamination problem. If it is chosen to drive the left and right patch devices 1a, 1b at different pressures, the left patch device 1a is even more exposed to the possibility of delamination since the left ventricle 21 is typically pressurised some six times higher than the right ventricle 22.

### OBJECT OF THE INVENTION

It is the object of the present invention to provide an improved implantable direct cardiac compression device which overcomes or substantially ameliorates at least one of the above described problems.

### SUMMARY OF THE INVENTION

There is disclosed herein an implantable direct cardiac compression device having a body comprising a flexible frontal cardiac compression wall and a rear wall together defining a pressurisable chamber. The cardiac compression wall is adapted to be affixed to the wall of a ventricle of a heart and to compress the ventricle upon pressurisation of the chamber. The rear wall is stiffer than the cardiac compression wall. The device is provided with two flexible flaps, one extending from each of two opposing lateral sides of the body and adapted to be affixed to the ventricle wall.

Typically, the cardiac compression wall and flaps each have a surface layer formed of a biointegratable material for affixing to the ventricle wall by biointegrating with the ventricle wall. Each of the flaps is preferably able to be trimmed with the use of scissors or the like.

Typically, the device is adapted to be affixed to the left ventricle of a heart.

In one form, the flaps each comprise the flap surface layer and a reinforcing layer secured to the flap surface layer for suturing to the pericardium encasing the heart.

There is further disclosed herein a method of treating a failing heart. In the method, an incision is created through the chest of a patient to be treated. The incision extends through the pericardium of the patient. A left implantable direct cardiac compression (DCC) device is introduced through the incision into the pericardial space of the patient. The left DCC device has a body comprising a flexible frontal cardiac compression wall and a rear wall together defining a pressurisable chamber. The left DCC device rear wall is stiffer than the left DCC device cardiac compression wall. The left DCC device is provided with two flexible flaps, one extending from each of two opposing lateral sides of the left DCC device body. A right direct cardiac compression (DCC) device is also introduced through the incision into the pericardial space of the patient. The right DCC device has a body comprising a flexible frontal cardiac compression wall and a rear wall together defining a pressurisable chamber. The right DCC device rear wall is stiffer than the right DCC device cardiac compression wall. The right DCC device cardiac compression wall is secured to the right ventricle of the heart. The left DCC device cardiac compression wall and flaps are secured to the left ventricle of the heart. The chamber of each of the left and right DCC devices is periodically pressurised to assist contraction of the left and right ventricles during systole.

Preferably, the method further comprises the step of securing each of the flaps to the pericardium on opposing sides of the incision through the pericardium.

The right DCC device cardiac compression wall and the left DCC device cardiac compression wall and flaps are typically secured to the right and left ventricles respectively by biointegrating with the right and left ventricles respectively.

There is further disclosed herein an implantable direct cardiac compression system. The system includes a left implantable direct cardiac compression (DCC) device having a body comprising a flexible frontal cardiac compression wall and a rear wall together defining a pressurisable chamber. The left DCC device cardiac compression wall is adapted to be affixed to the left ventricle of a heart and to compress the left ventricle upon pressurisation of the left DCC device chamber. The left DCC device rear wall is stiffer than the left DCC device cardiac compression wall.

The system further includes a right implantable direct cardiac compression (DCC) device having a body comprising a flexible frontal cardiac compression wall and a rear wall together defining a pressurisable chamber. The right DCC device cardiac compression wall is adapted to be affixed to the right ventricle of the heart and to compress the right ventricle upon pressurisation of the right DCC device chamber. The right DCC device rear wall is stiffer than the right DCC device cardiac compression wall.

The body of one of the DCC devices is provided with at least one strap extending from opposing lateral sides of the body and adapted to extend around the heart and the body of the other of the DCC devices in use.

Typically, the right DCC device comprises the one DCC device and the left DCC device comprises the other DCC device. The left DCC device is typically provided with one or more eyelet means adapted to receive the straps. The straps may be formed of a bioabsorbable material. The right DCC device is preferably provided with two straps each extending from each lateral side of the body.

Typically, the cardiac compression wall of each DCC device has a surface layer formed of a biointegratable material for affixing to the respective ventricle wall by biointegrating with the respective ventricle wall.

Preferably, the left DCC device is provided with two flexible flaps, one extending from each opposing lateral side of the body and adapted to be fixed to the left ventricle wall. Each of the flaps is able to be trimmed with the use of scissors or the like. The flaps typically each have a surface layer formed of a biointegratable material for affixing to the left ventricle wall by biointegrating with the left ventricle wall.

There is further disclosed herein another method of treating a failing heart. In this method an incision is created through the chest of a patient to be treated. The incision extends through the pericardium of the patient. A left implantable direct cardiac compression (DCC) device is introduced through the incision into the pericardial space of the patient. The left DCC device has a body comprising a flexible frontal cardiac compression wall and a rear wall together defining a pressurisable chamber. The left DCC device rear wall is stiffer than the left DCC device cardiac compression wall. A right direct cardiac compression (DCC) device is also introduced through the incision into the pericardial space. The right DCC device has a body comprising a flexible frontal cardiac compression wall and a rear wall together defining a pressurisable chamber. The right DCC device rear wall is stiffer than the right DCC device cardiac compression wall. The right DCC device is provided with at least one strap extending from opposing lateral sides of the right DCC device body. The right DCC device cardiac compression wall is positioned against the right ventricle of the heart. The left DCC device cardiac compression wall is positioned against the left ventricle of the heart. The straps are extended around the heart and the left DCC device body. The straps are fastened to thereby secure the left and right DCC devices to the left and right ventricles respectively. The chamber of each of the left and right DCC devices is periodically pressurised to assist contraction of the left and right ventricles during systole.

Preferably, the straps are threaded through eyelets provided on the left DCC device.

The left and right DCC devices are typically further secured to the left and right ventricles by biointegration of the cardiac compression walls with the ventricles.

Preferably, the left DCC device is provided with two flexible flaps one extending from each opposing lateral side of the body, the flaps being secured to the left ventricle.

In one preferred form, the flaps are trimmed prior to being introduced.

### BRIEF DESCRIPTION OF THE DRAWINGS

Preferred forms of the present invention will now be described by way of example with reference to the accompanying drawings wherein:

FIG. 1 is a front perspective view of a patch device of a prior art HeartPatch Pump.

FIG. 2 is a front perspective view of a heart having a HeartPatch Pump affixed.

FIG. 3 is a cross-sectional view of a left ventricle in diastole with a non-pressurised patch device of a prior art HeartPatch Pump affixed.

FIG. 4 is a cross-sectional view of the left ventricle and patch device of FIG. 3 with the ventricle in systole and the patch device in a pressurised state.

FIG. 5 is a front perspective view of a left direct cardiac compression device.

FIG. 6 is a rear perspective view of the left direct cardiac compression device of FIG. 5.

FIG. 7 is a cross-sectional view of the left direct cardiac compression device of FIG. 5 in a non-pressurised state.

FIG. 8 is a cross-sectional view of an alternate left direct cardiac compression device in a non-pressurised state.

FIG. 9 is a rear perspective view of a right direct cardiac compression device.

FIG. 10 is a front perspective view of a heart having the left direct cardiac compression device of FIG. 5 and the right direct cardiac compression device of FIG. 9 attached.

FIG. 11 is a cross-sectional view of the left ventricle in systole having a left direct cardiac compression device of FIG. 5 in a pressurised state affixed.

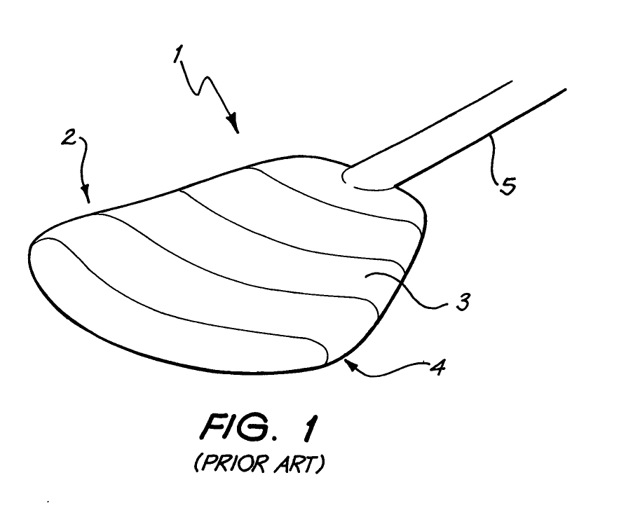

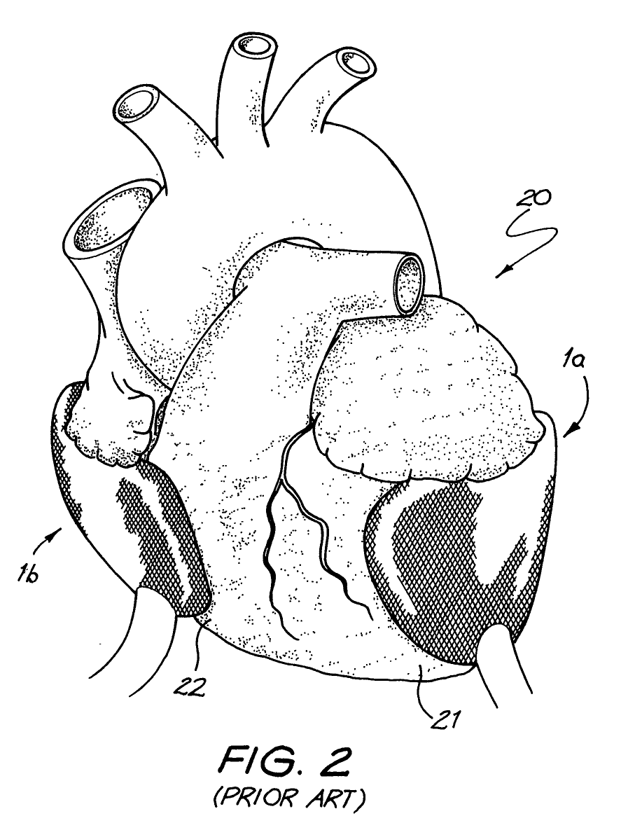

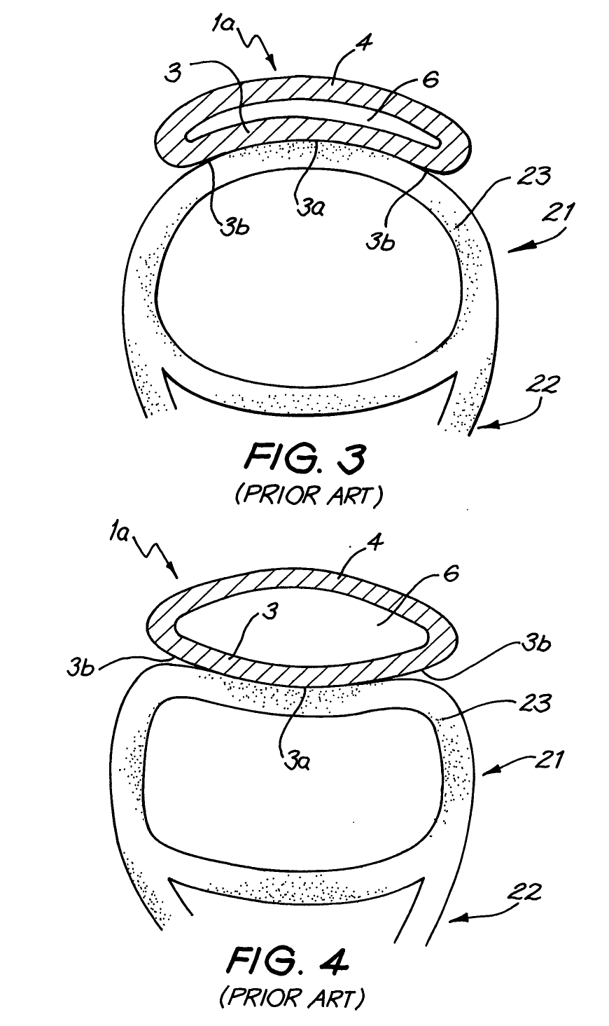

### DETAILED DESCRIPTION OF THE PREFERRED EMBODIMENTS

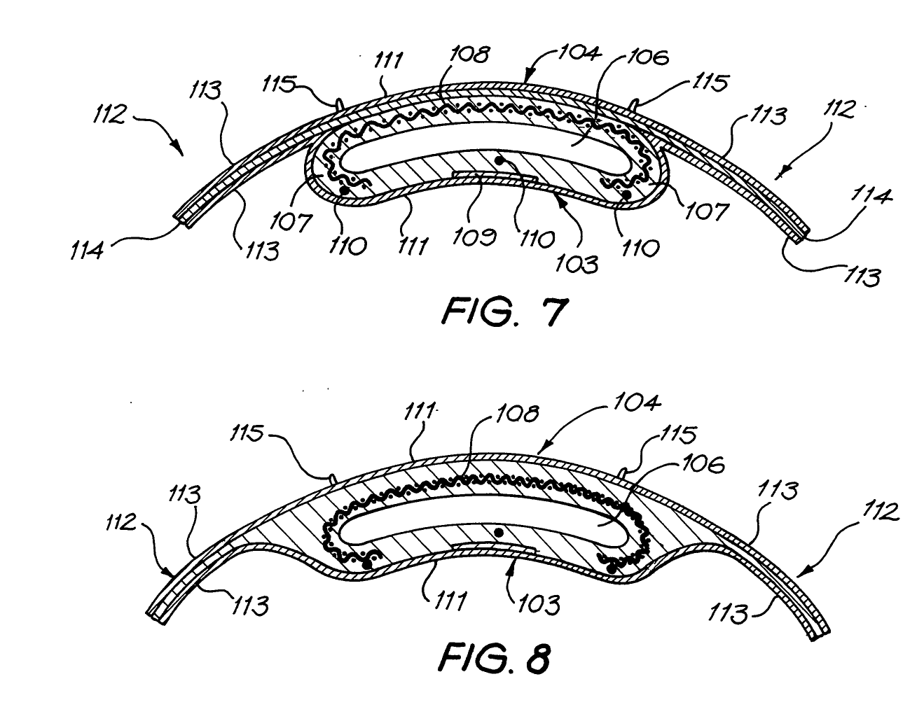

Referring to FIGS. 5 to 7 of the accompanying drawings, a left direct cardiac compression (DCC) device 101 has a body 102 having the same general form as that of the prior art HeartPatch Pump as disclosed in International Publication No. WO 00/78375. The body 102 has a flexible frontal cardiac compression wall 103 and a rear wall 104. The rear wall 104 is stiffer (i.e. less flexible) than the cardiac compression wall 103. The cardiac compression and rear walls 103, 104 are joined around their peripheral edges 107 so as to define a pressurisable chamber 106 between the walls. The body 102 is typically moulded in a single piece from polyurethane, silicone or a similar inert-flexible material. With the cardiac compression and rear walls 103, 104 thus being formed of the same material, a reinforcing mesh 108, which may conveniently be formed of teflon, nylon or the like, may be incorporated within the thickness of the rear wall 104 and extends through the peripheral portions 107, as best depicted in FIG. 7. The reinforcing mesh 108 serves to stiffen the rear wall 104 and the peripheral edge portions 107 such that the rear wall 104 is stiffer than the cardiac compression wall, resulting in pressurisation of the body chamber 106 acting to primarily deform the cardiac compression wall 103 against the left ventricular wall 23 as opposed to outwardly deforming the rear wall 104. The body 102 will also typically be provided with an ECG electrode 109 and sonomicrometer sensors 110, again in the same manner as the prior art HeartPatch Pump.

As best depicted in FIG. 7, the cardiac compression and rear walls 103, 104 include a surface layer of biointegratable material 111 bonded to the polyurethane/silicone main thickness of the walls 103, 104, again as per the prior art HeartPatch Pump. The biointegratable material 111 enables biointegration of the cardiac compression wall 103 with the left ventricular wall 23, and also greatly reduces the chance of fibrous capsule formation and infection resulting from foreign material rejection. Such fibrous capsule formation would otherwise likely stiffen the ventricular wall and constrict normal functioning of the same.

The body 102 is curved about a longitudinal axis thereof corresponding generally to the longitudinal axis of the left ventricle 21, a concave cardiac compression wall 103 is thus provided, which, when the device is in a non-pressurised state, approximates the curvature of the left ventricular wall 23 in diastole. A tube 105 extends from the end of the body 102 and communicates with the chamber 106, again in the same manner as the prior art HeartPatch Pump.

The left DCC device 101 is provided with two flexible flaps 112, one extending from each lateral side of the body 102. The flaps 112 are adapted to be affixed to the left ventricular wall 23, here by way of being provided with a surface layer 113 formed of a biointegratable material which biointegrates with the left ventricular wall 23 in the same manner as the surface layer 111 of the cardiac compression wall 103.

As it is desired to suture the flaps 112 to the pericardium (as will be discussed below), and currently available biointegratable materials do not provide sufficient strength for load-bearing sutures, the flaps 112 will also typically be provided with a fabric reinforcing layer 114, which may be conveniently formed of nylon, to act as a load-bearing layer. The flap biointegratable surface layer 113 will preferably be bonded to each opposing side of the reinforcing fabric layer 114 so as to inhibit fibrous capsule formation.

The flaps 112 may be formed separately to the body 102 and bonded thereto, or alternatively the flaps 112 may be formed integrally with the body 102 by any of various methods. Referring to FIG. 7 for example, the biointegratable surface layers 111, 113 of the body and flaps may be formed as two sheets of biointegratable material, one extending across the front face of the device from the edge of one flap 112 across the cardiac compression wall 103 and across the opposing flap 112, and the other extending across the flaps 112 and rear wall 104. The fabric reinforcing layer 114 may also extend from one flap 112 across the rear wall 104 and to the opposing flap 112. The materials used in the flaps 112 are selected such that the flaps may be readily trimmed with scissors, a surgical knife or the like by a surgeon prior to implantation.

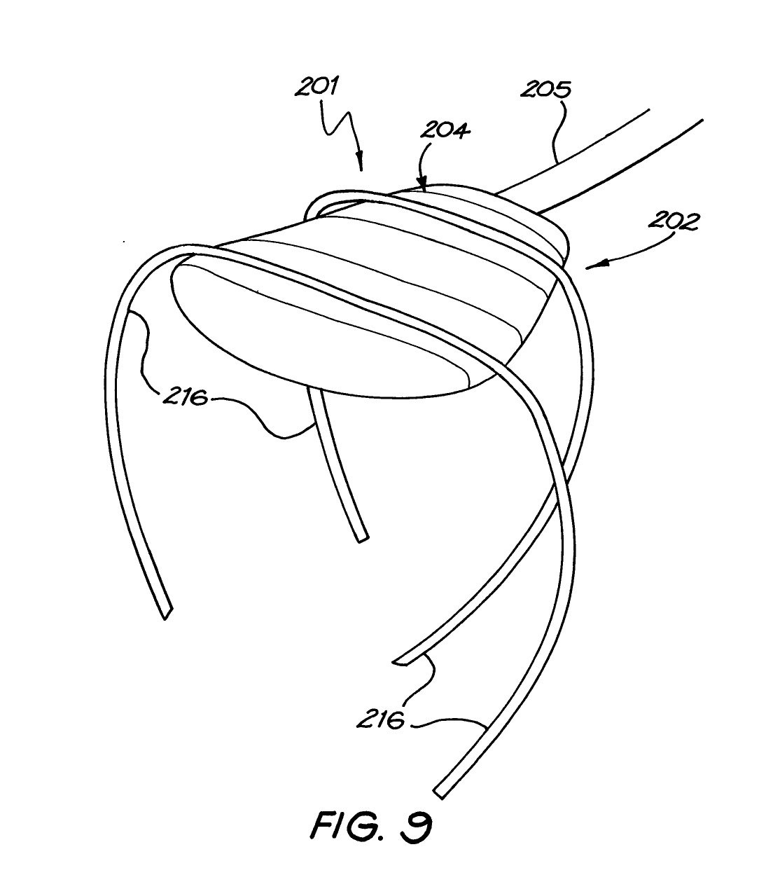

In another form, as depicted in FIG. 8, the flaps 112 may be formed as an integral continuation of the polyurethane/silicone moulding of the body, again with a layer of biointegratable material forming a surface layer 111, 113 extending across each face of the flaps 112 and the cardiac compression and rear walls 103, 104.

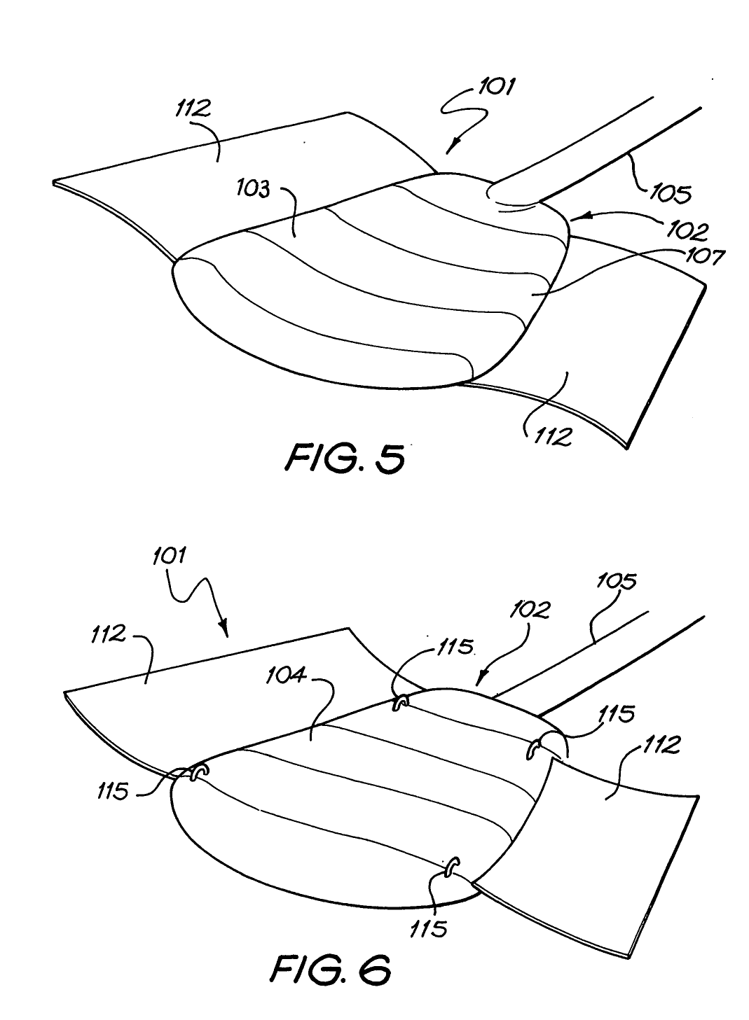

The rear wall 104 of the left DCC device body 102 is also provided with a series of four eyelets 115 spaced along the lateral side edges of the body as depicted in FIG. 6. These eyelets 115 may be integrally moulded from polyurethane/silicone with the rear wall 104, or may be separately formed and secured to the rear wall 104. The purpose of these eyelets 115 will be discussed below.

Referring to FIG. 9, a right direct cardiac compression (DCC) device 201 again has a body 202 again of the same general form as that of the prior art HeartPatch Pump disclosed in International Publication No. WO 00/78375 and as discussed above in relation to the left DCC device 101. The right DCC device 201, however, will typically not be provided with flaps as opposed to the left DCC device 101. The size and shape of the body 202 of the right DCC device 201 also differs. from that of the left DCC device 101 so as to fit the right ventricle 22.

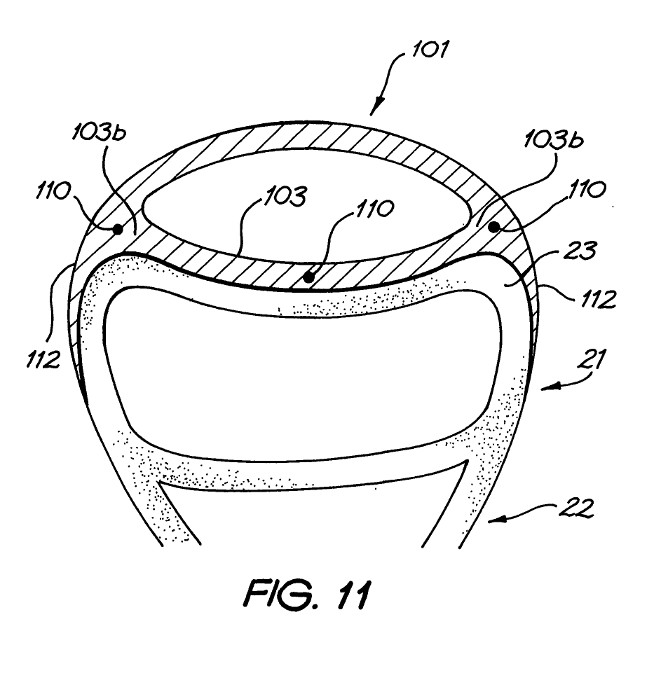

Also, rather than being provided with eyelets as for the left DCC device 101, the right DCC device 201 is provided with one or more, typically two, straps 216 extending from each lateral side of the body 202. The straps 216 provide for acute attachment of the left and right DCC devices 101, 201 as will be discussed below. The straps 216 may be separately bonded to each opposing side of the body 202, or may consist of two straps 216 each extending across and bonded to the rear wall 204 as depicted in FIG. 11. Rather than being bonded to the rear wall 204, the straps 216 may be built into the thickness of the rear wall 204 during moulding of the rear wall 204. The straps 216 will preferably be formed of a bioabsorable material, such as Vycral™.

The bioabsorable straps 216 will be completely absorbed after 3 months, by which time biointegration of the left and right DCC devices 101, 201 with the heart will have been well formed. The straps 216 will retain most of their strength over the first couple of weeks whilst biointegration of the DCC devices 101, 201 takes place.

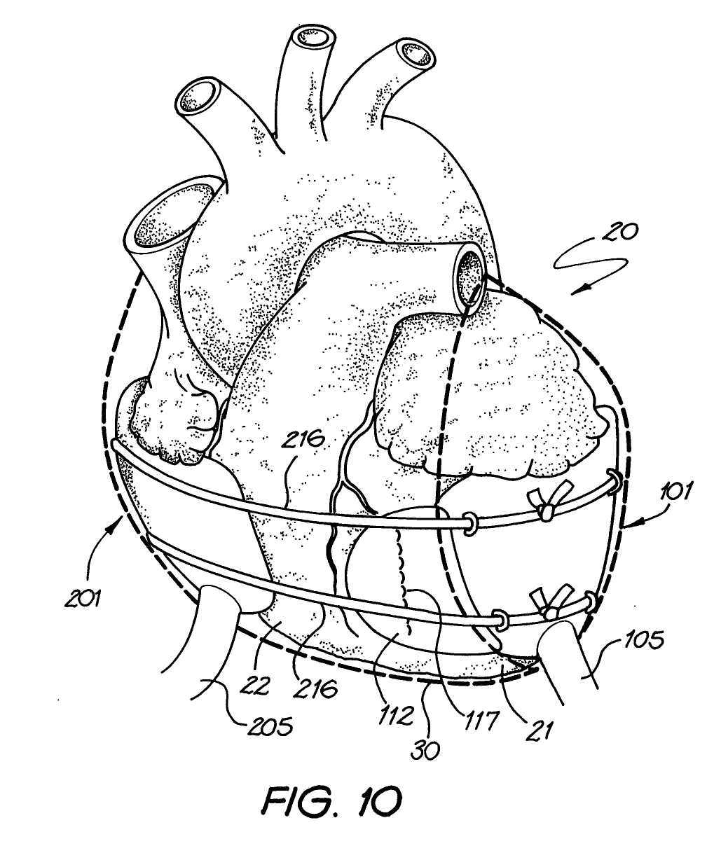

Implantation of the left and right DCC devices 101, 201 to treat a failing heart 20 will now be described with particular reference to FIG. 10.

An incision is firstly made through the chest wall of the patient, with such incision extending through the pericardium 30 surrounding the heart 20. The left and right DCC devices 101, 201 are then introduced through the incision and positioned in place beside the left and right ventricles 21, 22 respectively. With the right ventricle 22 being generally positioned on the posterior side of the heart 20, the right DCC device 201 will typically be positioned in place first, with its cardiac compression wall 203 engaging the right ventricle 22. The intact rear portion of the pericardium 30 will assist in holding the right DCC device 201 in place. A stay stitch may also be used to temporarily hold the right DCC device in place against the right ventricle 22. The right DCC device 201 is positioned such that the straps 216 are free to extend around the front of the heart 20.

The left DCC device 101 is then positioned over the left ventricle 201, with its cardiac compression wall 103 and flaps 112 engaging the left ventricle 21. Dependant upon the size of the heart 20, and particularly the size of the left ventricle 21, the surgeon may trim the flaps 112 to size as deemed appropriate prior to introduction of the left DCC device 101. The rimmed flaps 112 should typically remain widest over the mid-length thereof where the maximum inflation of the body 102 occurs.

With the left and right DCC devices 101, 201 in place, the straps 216 are guided over the flaps 112 and through the eyelets 115 provided on the left DCC device body 102. The straps 216 are then lightly tensioned, sufficient to hold the left and right DCC devices 101, 201 in place but without creating any significant constriction against ventricular relaxation, and tied off. The straps 216 are typically tied off adjacent the left DCC device rear wall 104.

To further secure the left DCC device 101 in place, the flaps 112 may be sutured to the pericardium 30, on opposing sides of the incision created, by way of sutures 117. The sutures 117 may be bioabsorbable, absorbing once biointegration of the left and right DCC devices 101,201 is complete. Alternatively the sutures may be non-absorbable such that the left DCC device 201 remains secured to the pericardium. In any event, if the rear wall 104 and rear face of the flaps 112 are provided with a biointegratable surface layer, then the left DCC device 101 will biointegrate with the pericardium over time, again fixing the left DCC device 101 to the pericardium 30. Rather than suturing the flaps 112 to the pericardium 30, it is envisaged that tissue glue such as Cardial™ may be used as an alternative to bond the flaps 112.

The chambers 106, 206 of the left and right DCC devices 101, 201 are coupled to a pressurisation and control mechanism by way of the tubes 105, 205 extending through the incision created in the chest of the patient, in the same manner as the prior art HeartPatch Pump. The pressurisation and control mechanism may be configured to pressurise the chambers 106, 206 to equal pressures. Alternatively, the pressurisation and control mechanism may be configured to pressurise the chamber 206 of the right DCC device 201 to a lower pressure than that applied to the chamber 106 of the left DCC device 101, given the lower pressure in the right ventricle 22.

The straps 216 provide acute attachment of the left and right DCC devices 101, 201, enabling immediate activation of the devices by pressurisation using the external pressurisation and control mechanism. The straps 216 provide adequate acute attachment whilst only providing a minor constriction across two discrete portions of the left and right ventricles 21, 22 over which the straps 216 pass. The remainder of the left and right ventricles 21, 22 are essentially unrestrained.

As well as providing an increased surface area for biointegration of the left DCC device 101, thereby providing sufficient biointegration for operation of the device without need for the straps 216 in a shorter period, and enabling acute attachment to the pericardium 30, the flaps 112 of the left DCC device 101 also alleviate the delamination problem discussed above.

Referring to FIG. 11 depicting the left ventricle 21 in systole with the left DCC device 101 in a pressurised state, the flaps 112 act to anchor the lateral edges 103b of the cardiac compression wall 103, more evenly distributing the peel stresses normally encountered towards the edges 103b of the cardiac compression wall 103, across the breadth of the flaps 112. As a result, substantially the entire cardiac compression wall 103 acts to compress the left ventricular wall 23, providing a much more even compression with reduced bowing of the device, as compared to the prior art HeartPatch Pump, to more evenly assist ventricular contraction during systole without significant unusual deformation. Preventing delamnination of the cardiac compression wall edges 103b also ensures that the sensors 110 embedded in the cardiac compression wall 103 remain fixed in relation to the left ventricular wall 23. Such fixation enables accurate measurement to be taken by the sensors 110, particularly of ventricular volume change achieved by inflation of the left DCC device 101 used by the control mechanism to optimise the pressurisation cycle.

If desired, flaps may also be provided on the right DCC device 201 should the chambers 106, 206 of both the left and right DCC devices 101, 201 be pressurised to the same pressure. However given the possibility of using much lower pressures to effectively pressurise the chamber 206 of right DCC device 201 and the consequent lower potential for delamination, such flaps will typically not be necessary on the right DCC device 201.

1. An implantable direct cardiac compression device comprising:
a body having a flexible frontal cardiac compression wall and a rear wall together defining a pressurisable chamber, said cardiac compression wall being adapted to be affixed to the wall of a ventricle of a heart and to compress the ventricle upon pressurisation of said chamber, said rear wall being stiffer than said cardiac compression wall, said body having two opposing lateral sides; and
two flexible flaps, one of said flaps extending from one of said lateral sides of said body and the other of said flaps extending from the other of said lateral sides of said body, said flaps being adapted to be affixed to the ventricle wall.

2. The device of claim 1 wherein said cardiac compression wall and said flaps each have a surface layer formed of a biointegratable material for affixing to the ventricle wall by biointegrating with the ventricle wall.

3. The device of claim 1 wherein each of said flaps is able to be trimmed is with the use of scissors or the like.

4. The device of claim 1 wherein said cardiac compression wall is adapted to be affixed to the left ventricle of a heart.

5. The device of claim 2 wherein said flaps each comprise said flap surface layer and a reinforcing layer secured to said flap surface layer for suturing to the pericardium encasing the heart.

6. A method of treating a failing heart comprising the steps of:
creating an incision through the chest of a patient to be treated, said incision extending through the pericardium of the patient;
introducing a left implantable direct cardiac compression (DCC) device through said incision into the pericardial space of the patient, said left DCC device having a body comprising a flexible frontal cardiac compression wall and a rear wall together defining a pressurisable chamber, said left DCC device rear wall being stiffer than said left DCC device cardiac compression wall, said left DCC device further having two flexible flaps, one said flap extending from one lateral side of said left DCC device body and the other of said flaps extending from an opposing lateral side of said left DCC device body;
introducing a right direct cardiac compression ACC) device through said incision into the pericardial space of the patient, said right DCC device having a body comprising a flexible frontal cardiac compression wall and a rear wall together defining a pressurisable chamber, said right DCC device rear wall being stiffer than said right DCC device cardiac compression wall;
securing said right DCC device cardiac compression wall to the right ventricle of the heart;
securing said left DCC device cardiac compression wall and flaps to the left ventricle of the heart;
periodically pressurising said chamber of each of said left and right DCC devices to assist contraction of the left and right ventricles during systole.

7. The method of claim 6 further comprising the step of securing each of said flaps to the pericardium on opposing sides of said incision.

8. The method of claim 6 wherein said right DCC device cardiac compression wall and said left DCC device cardiac compression wall and flaps are secured to the right and left ventricles respectively by biointegrating with the right and left ventricles respectively.

9. An implantable direct cardiac compression system comprising:
a left implantable direct cardiac compression (DCC) device having a body comprising a flexible frontal cardiac compression wall and a rear wall together defining a pressurisable chamber, said left DCC device cardiac compression wall being adapted to be affixed to the left ventricle of a heart and to compress the left ventricle upon pressurisation of said left DCC device chamber, said left DCC device rear wall being stiffer said left DCC device cardiac compression wall;
a right implantable direct cardiac compression (DCC) device having a body comprising a flexible frontal cardiac compression wall and a rear wall together defining a pressurisable chamber, said right DCC device cardiac compression wall being adapted to be affixed to the right ventricle of the heart and to compress the right ventricle upon pressurisation of said right DCC device chamber, said right DCC device rear wall being stiffer than said right DCC device cardiac compression wall;
wherein said body of one of said DCC devices is provided with at least one strap extending from opposing lateral sides thereof and adapted to extend around the heart and said body of the other of said DCC devices in use.

10. The system of claim 9 wherein said right DCC device comprises said one DCC device and said left DCC device comprises said other DCC device.

11. The system of claim 10 wherein said left DCC device is provided with one or more eyelets adapted to receive said straps.

12. The system of claim 9 wherein said straps are formed of a bioabsorbable material.

13. The system of claim 10 wherein said right DCC device is provided with two said straps each extending from each lateral side of said right DCC device body.

14. The system of claim 9 wherein said cardiac compression wall of each said DCC device has a surface layer formed of a biointegratable material for affixing to the respective ventricle wall by biointegrating with the respective ventricle wall.

15. The system of claim 9 wherein said left DCC device is provided with two flexible flaps, one of said flaps extending from a lateral side of said body and the other said flaps extending from an opposing lateral side of said body, said flaps adapted to be fixed to the left ventricle wall.

16. The system of claim 15 wherein each of said flaps is able to be trimmed with the use of scissors or the like.

17. The system of claim 15 wherein said flaps each have a surface layer formed of a biointegratable material for affixing to the left ventricle wall by biointegrating with the left ventricle wall.

18. A method of treating a failing heart comprising the steps of:
creating an incision through the chest of a patient to be treated, said incision extending through the pericardium of the patient;
introducing a left implantable direct cardiac compression (DCC) device through said incision into the pericardial space of the patient, said left DCC device having a body comprising a flexible frontal cardiac compression wall and a rear wall together defining a pressurisable chamber, said left DCC device rear wall being stiffer than said left DCC device cardiac compression wall;
introducing a right direct cardiac compression (DCC) device through said incision into the pericardial space of the patient, said right DCC device having a body comprising a flexible frontal cardiac compression wall and a rear wall together defining a pressurisable chamber, said right DCC device rear wall being stiffer than said right DCC device cardiac compression wall, said right DCC device being provided with at least one strap extending from opposing lateral sides of said right DCC device body;
positioning said right DCC device cardiac compression wall against the right ventricle of the heart;
positioning said left DCC device cardiac compression wall against the left ventricle of the heart,
extending said strap(s) around the heart and the left DCC device body;
fastening said strap(s) to secure said left and right DCC devices to the left and right ventricles respectively; and
periodically pressurising said chamber of each of said left and right DCC devices to assist contraction of the left and right ventricles during systole.

19. The method of claim 18 wherein said strap(s) is/are threaded through at least one eyelet provided on the left DCC device.

20. The method of claim 18 wherein said left and right DCC devices are further secured to the left and right ventricles by biointegration of said cardiac compression walls with the ventricles.

21. The method of claim 18 wherein said left DCC device is provided with two flexible flaps, one said flap extending from a lateral side of said left DCC device body, the other said flap extending from an opposing lateral side of said left DCC body, said method further comprising the step of securing said flaps to the left ventricle.

22. The method of claim 18 wherein said flaps are trimmed prior to being introduced.
